(19) United States
(12) Patent Application Publication
(10) Pub. No.: US 2025/0161225 A1
(43) Pub. Date: May 22, 2025

(54) FUMARIC ACID COMPOSITIONS WITH INCREASED BIOAVAILABILITY AND REDUCED SIDE EFFECTS

(71) Applicant: Vitalis LLC, Wilmington, DE (US)

(72) Inventor: Joseph Habboushe, New York, NY (US)

(21) Appl. No.: 18/823,016

(22) Filed: Sep. 3, 2024

Related U.S. Application Data
(63) Continuation of application No. 17/275,071, filed on Mar. 10, 2021, now abandoned.
(60) Provisional application No. 62/729,063, filed on Sep. 10, 2018.

(51) Int. Cl. A61K 9/50; A61K 9/00; A61K 31/192; A61K 31/194; A61K 31/225; A61P 29/00

(52) U.S. Cl. CPC A61K 9/5084; A61K 9/0053; A61K 31/192; A61K 31/194; A61K 31/225; A61P 29/00

(57) ABSTRACT

Provided are compositions and methods for treating multiple sclerosis (MS). One embodiment of the disclosed method entails orally administering to a MS patient a first amount of a nonsteroidal anti-inflammatory drug (NSAID), such as aspirin, and a second amount of fumaric acid or an ester or a salt thereof. The NSAID is administered at from about 80 mg to about 500 mg per day and the fumaric acid or ester or salt thereof is administered at about 360 to about 420 mg per day. The compositions are formulated so that, upon oral administration to a subject, the both the NSAID and the fumaric acid or ester or salt thereof are released in the gastrointestinal track of the subject, and the NSAID is released at substantially the same time as, slower than, or later than the fumaric acid or ester or salt thereof. The delayed release of NSAID, it is herein observed, increased the bioavailability of the fumaric acid or ester or salt thereof.

### CROSS-REFERENCE TO RELATED APPLICATIONS

This application claims the benefit under 35 U.S.C. § 119 of U.S. Application No. 62/729,063, filed Sep. 10, 2018, the contents of which is incorporated herein by reference by its entirety.

### BACKGROUND

Fumaric acid and its ester or salt, such as dimethyl fumarate (DMF), monomethyl fumarate (MMF), are useful for treating various diseases such as multiple sclerosis (MS) and psoriasis.

Multiple sclerosis (MS) is the most common autoimmune disorder affecting the central nervous system. In 2013, about 2.3 million people were affected globally with rates varying widely in different regions and among different populations. About 20,000 people died from MS in 2013, up from 12,000 in 1990. MS is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. MS takes several forms, with new symptoms either occurring in isolated attacks (relapsing forms) or building up over time (progressive forms). Between attacks, symptoms may disappear completely; however, permanent neurological problems often remain, especially as the disease advances.

Psoriasis is a long-lasting autoimmune disease characterized by patches of abnormal skin. These skin patches are typically red, dry, itchy, and scaly. On people with darker skin the patches may be purple in color. Psoriasis varies in severity from small, localized patches to complete body coverage. Injury to the skin can trigger psoriatic skin changes at that spot, which is known as the Koebner phenomenon.

Both DMF and MMF activate the nuclear-factor-E2-related factor-2 (Nrf2) transcriptional pathway, which induces anti-inflammatory and neuroprotective modalities in MS and psoriasis patients. The approved dosage of DMF for treating MS is 480 mg daily. About 30% to 40% of treated individuals, however, suffer from cutaneous flush which is associated with both DMF and MMF. Such adverse effects, therefore, limit the use of DMF and MMF.

### SUMMARY

The instant inventor made the unexpected discovery that when aspirin, a nonsteroidal anti-inflammatory drug (NSAID), is co-administered with a fumaric acid or its ester or salt, such as DMF, the bioavailability of the fumaric acid or its ester or salt is increased by at least about 5% when both are released in the GI track and when the NSAID is released at substantially the same time as. or more slowly than or after the fumaric acid or its ester or salt. The increased bioavailability of the fumaric acid or its ester or salt is believed to increase its efficacy and decrease the commonly associated side effects such as flushing. Moreover, when the formulation further includes an intraorally dissolved NSAID, the intraorally absorbed NSAID can further reduce the flushing side effect. The currently approved dosage for DMF to treating MS is 480 mg daily. With the presently disclosed formulations, a 360-420 mg daily administration would be therapeutically sufficient.

Accordingly, the present disclosure provides treatment regimens for diseases that can be suitably treated with fumaric acid or its ester or salt, such as dimethyl fumarate (DMF), monomethyl fumarate (MMF), or the combination thereof. Examples of such diseases include multiple sclerosis (MS), psoriasis, necrobiosis lipoidica, granuloma annulare, sarcoidosis, granulomatous and inflammatory skin disorders, lichen planus pityriasis rubra pilaris, chronic discoid lupus erythematosus, cheilitis granulomatosa, annular elastotic giant cell granuloma, malign melanoma, lupus erythematosus, aplopecia areata, hidradenitis suppurativa, other granulomatous and inflammatory skin disorders, other inflammatory disorders such as colitis, DNA damage in tumor, gastrointestinal ulceration, collagen type II degradation, and other immune modulated diseases. In some embodiments, the treatment methods enable the effective use of a daily dose of fumaric acid or an ester or salt thereof that is lower than their recommended use (e.g., 480 mg per day), without compromise of the treatment outcome.

In one embodiment, a dosage form is provided, comprising a first portion comprising a adjuvant designed to increase the bioavailability of fumaric acid, such as a nonsteroidal anti-inflammatory drug (NSAID) (e.g., aspirin); and a second portion comprising a fumaric acid or an ester or a salt thereof, wherein the first portion and the second portion are formulated such that, upon oral administration to a subject, the first portion and the second portion are released in the gastrointestinal track of the subject, and the NSAID in the first portion is released at substantially the same time as. slower than or later than the fumaric acid or ester or salt thereof in the second portion.

In one embodiment, a dosage form is provided, comprising a first portion comprising aspirin; and a second portion comprising a fumaric acid or an ester or a salt thereof, wherein the first portion and the second portion are formulated such that, upon oral administration to a subject, the first portion and the second portion are released in the gastrointestinal track of the subject, and the aspirin in the first portion is released slower or later than the fumaric acid or ester or salt thereof in the second portion.

In some embodiments, the nonsteroidal anti-inflammatory drug (NSAID), such as aspirin, and fumaric acid or ester or salt thereof are each individually formulated as enterically coated microspheres.

In some embodiments, the NSAID and fumaric acid or ester or salt thereof are each individually formulated as enterically coated microspheres, and wherein the enteric coating of the NSAID is stronger or thicker than the enteric coating of the fumaric acid or ester or salt thereof.

In some embodiments, the NSAID in the first portion is released at substantially the same time as the fumaric acid or ester or salt thereof in the second portion. In some embodiments, the NSAID in the first portion is released at least 1 minute, or at least 2, 3, 4, 5, 6, 7, 8, 9, 10, 11, 12, 13, 14, 15, 16, 17, 18, 19 or 20 minutes after the release of the fumaric acid or ester or salt thereof in the second portion.

In some embodiments, the fumaric acid or ester or salt thereof is dimethyl fumarate. In some embodiments, the dosage form comprises from about 180 mg to about 210 mg (e.g., 195, 196, 197, 198 or 199 mg) of dimethyl fumarate. In some embodiments, the first portion comprises from about 20 mg to about 500 mg of aspirin.

In some embodiments, provided is an enteric coated capsule comprising the dosage form of the disclosure. In some amendments, the capsule further comprises a third portion comprising a nonsteroidal anti-inflammatory drug (NSAID), such as aspirin, formulated to dissolve in an oral cavity of a subject. In some amendments, the third portion comprises from about 20 mg to about 500 mg aspirin.

In addition to multiple sclerosis and psoriasis, fumaric acid or ester or salt thereof can also be used for treating other diseases and conditions such as motor neuron disease, neurodegenerative diseases, autoimmune diseases, inflammatory diseases, sepsis, and skin diseases or conditions.

A motor neuron disease a neurological condition that selectively affects motor neurons. Examples include amyotrophic lateral sclerosis (ALS), hereditary spastic paraplegia (HSP), primary lateral sclerosis (PLS), progressive muscular atrophy (PMA), progressive bulbar palsy (PBP) and pseudobulbar palsy.

Neurodegenerative diseases are results of progressive loss of structure or function of neurons, including death of neurons. Examples include amyotrophic lateral sclerosis, Parkinson's, Alzheimer's, and Huntington's, which occur as a result of neurodegenerative processes.

Non-limiting examples of autoimmune or inflammatory disease include Parkinson's disease, arthritis, rheumatoid arthritis, multiple sclerosis, psoriasis, psoriatic arthritis, Crohn's disease, inflammatory bowel disease, ulcerative colitis, lupus, systemic lupus erythematous, juvenile rheumatoid arthritis, juvenile idiopathic arthritis, Grave's disease, Hashimoto's thyroiditis, Addison's disease, celiac disease, dermatomyositis, multiple sclerosis, myasthenia gravis, pernicious anemia, Sjogren syndrome, type I diabetes, vasculitis, uveitis, atherosclerosis and ankylosing spondylitis.

Skin diseases are various skin problems, from small red bumps on the skin to widespread rashes. Some skin conditions can be unsightly but harmless, while others may be contagious. Many skin conditions are also itchy or painful. The presently disclosed compositions and methods are suitable for treating these diseases and the symptoms. Non-limiting examples of symptoms include itch, swelling, redness, rash, flaky, scaly skin, blisters, oozing and bumps or growths.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Relapse-remitting multiple sclerosis,” or RRMS, is a type of MS of which symptoms can appear suddenly and be severe and can then go quiet for months or years. Between flare-ups, the disease tends not to progress or progresses relatively slowly, and symptoms may disappear.

“Secondary-progressive multiple sclerosis,” or SPMS, is a MS condition in which the disease tends to progress steadily. This can happen with or without relapses. Many patients with RRMS may transition to SPMS at some point in the course of their disease.

“Fumaric acid” is the chemical compound with the formula HO₂CCH═CHCO₂H. The “salts and esters” of fumaric acid are known as fumarates or fumaric acid esters (FAE), and include any ester (e.g., mono ester hydrogen fumarate or salt thereof or diester of fumaric acid), such as dimethyl fumarate (DMF), monomethyl fumarate (MMF), diethyl fumarate, monoethyl fumarate, diroximel fumarate (previously BIIB098 and ALKS8700), etc. The fumaric acid can comprise a mixture of DMF, also three monoethyl hydrogen fumarates or salt thereof (calcium, magnesium, and zinc salts) (e.g., Fumaderm®). The fumaric acid can comprise ALKS 8700 (“a MMF molecule” which is a prodrug to MMF).

Dimethyl fumarate (DMF) is the dimethyl ester of fumaric acid, having a chemical name of dimethyl (E)-butenedioate. DMF and its metabolite, monomethyl fumarate (MMF), were initially recognized as effective hypoxic cell radiosensitizers. They are also used as oral therapy for psoriasis. Other diseases, such as necrobiosis lipoidica, granuloma annulare, and sarcoidosis may also be suitably treated with DMF and MMF.

In a non-medical setting, DMF is applied as a biocide to prevent growths of mold during storage or transport in a humid climate. However, due to incidences of allergic reactions after skin contact the European Union banned DMF in consumer products since 1998, and since January 2009 the import of products containing DMF was also banned. Medical use of DMF also is known to come with associated side effects, such as progressive multifocal leukoencephalopathy, which can be serious. Another side effect associated with the use of DMF or MMF is the flushing, which has been reported to cause non-compliance of patients.

A commercial form of DMF for treating MS is Tecfidera®. According to the drug label, the starting dose for Tecfidera® is 120 mg twice a day orally. After 7 days, the dose should be increased to the maintenance dose of 240 mg twice a day orally. Temporary dose reductions to 120 mg twice a day may be considered for individuals who do not tolerate the maintenance dose. Higher doses of Tecfidera® are not recommended.

It is a surprising and unexpected discovery of the instant inventor that administration of both aspirin and fumaric acid or its ester or salt such as DMF and MMF achieves increased bioavailability of the fumaric acid or its ester or salt when both are released in the GI track but the aspirin is released at substantially the same time as (i.e., within about 1 minute), more slowly than, or after the fumaric acid or its ester or salt. Such a dual administration, therefore, makes it possible to use a lower dose (e.g., 360-420 mg per day) of fumaric acid of the ester or salt thereof, as compared to the conventional commercial dose (e.g., 480 mg per day), to achieve the same efficacy as the conventional dose would but with greatly reduced side effects.

The impact of aspirin, a nonsteroidal anti-inflammatory drug (NSAID), on the bioavailability of DMF has been evaluated previously and acknowledged by the US FDA. In Sheikh et al., Clin Ther. 2013; 35:1582-94, for example, the authors observed that pretreatment with 325 mg aspirin for 4 days did not affect the pharmacokinetic profile of DMF (abstract). In other words, aspirin pretreatment did not change the bioavailability of the DMF. It necessarily follows that the present discovery that release of aspirin concurrently with, or after, DMF increased the bioavailability of DMF by about 5% is surprising and unexpected.

This discovery makes it possible to use a lower dose (e.g., 360-420 mg per day) while achieving the same or substantially similar efficacy as compared to the conventional commercial dose (e.g., 480 mg per day), to achieve the same efficacy as the conventional dose. The effect can be achieved with sequential administration or concurrent administration of two or more separate compositions, or administration of a composition that includes two or more different ingredients.

In certain embodiments, the nonsteroidal anti-inflammatory drug (NSAID) works by inhibiting the activity of cyclooxygenase enzymes (COX-1 and/or COX-2). In certain embodiments, the NSAID is aspirin. The NSAID can be non-selective and COX-2 selective. In certain embodiments, the NSAID is non-selective, and thus inhibits the activity of both COX-1 and COX-2. These NSAIDs, while reducing inflammation, also inhibit platelet aggregation (e.g., aspirin). Exemplary NSAIDs include, but are not limited to, aspirin, ibuprofen and naproxen.

In some embodiments, a co-formulation is disclosed. In some embodiments, the nonsteroidal anti-inflammatory drug (NSAID), such as aspirin, and fumaric acid of the ester or salt thereof are in separate portions in the co-formulation, such as a tablet.

### Example Co-Formulations

Pharmaceutical formulations are provided, in some embodiments. The formulations may include the nonsteroidal anti-inflammatory drug (NSAID), such as aspirin, at a suitable dose and form and a fumaric acid or an ester or a salt thereof at a suitable dose and form. In some embodiments, the formulation includes at last about 20, 30, 40, 50, 60, 70, 80, 90, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 mg aspirin. In some embodiments, the formulation includes not more than about 420, 410, 400, 390, 380, 370, 360, 350, 340, 330, 325, 320, 315, 310, 305, 300, 295, 290, 285, 280, 275, 260, 255, 250, 245, 240, 235, 230, 225, 220, 215, 210, 205, 200, 190, 180, 170, 160, or 150 mg aspirin.

In some embodiments, the formulation includes at least about 180, 185, 190, 195, 200, 205, 210, 360, 365, 370, 375, 380, 385, 390, 395, 400, 405, 410, 415, or 420 mg of a fumaric acid or an ester or a salt thereof. In some embodiments, the formulation includes not more than about 420, 410, 400, 390, 380, 370, 360, 210, 205, 200, 195, 190, 185, or 180 mg of a fumaric acid or an ester or a salt thereof. In some embodiments, the amount of the fumaric acid or an ester or a salt thereof is about 180, 185, 190, 195, 196, 197, 198, 199, 200, 205, or 210 mg.

In some embodiments, the fumaric acid or an ester or a salt thereof is dimethyl fumarate, optionally in combination with an additional fumaric acid or an ester or a salt thereof. In some embodiments, the additional fumaric acid or an ester or a salt thereof is monomethyl fumarate or a salt thereof (e.g., Na⁺, K⁺, Ca²⁺, Zn²⁺, Mg²⁺, Fe²⁺). In some embodiments, the monomethyl fumarate is hydrogen monomethyl fumarate. In some embodiments, the pharmaceutical composition consists essentially of an effective amount of aspirin and an effective amount of dimethyl fumarate. In some embodiments, the pharmaceutical composition consists essentially of an effective amount of a nonsteroidal anti-inflammatory drug (NSAID) and an effective amount of dimethyl fumarate.

In some embodiments, the pharmaceutical compositions described herein are formulated as a capsule comprising nonsteroidal anti-inflammatory drug (NSAID), such as aspirin, and a fumaric acid or an ester or a salt thereof, wherein the NSAID and fumaric acid or an ester or a salt thereof are each formulated as an enterically coated microsphere contained within a capsule shell. The microspheres described herein may also include non-spherical microparticles, such as oblong or cylindrical microparticles. The enteric coating of the microspheres containing the nonsteroidal anti-inflammatory drug (NSAID), such as aspirin, however, may be stronger or thicker than the enteric coating of the microspheres containing the fumaric acid or ester or salt thereof.

In some embodiments, the enteric coatings are formulated such that the API (NSAID or fumaric acid or an ester or a salt thereof (e.g., DMF)) is released in the gastrointestinal tract (e.g., the small intestine). In some embodiments, the enteric coatings on the microspheres are formulated or applied such that the NSAID is released in the gastrointestinal tract (e.g., the small intestine) just after (e.g., 1-5, 1-10, 1-15, or 1-20 minutes) the fumaric acid or an ester or a salt thereof (e.g., DMF). Accordingly, in some embodiments, the enteric coating on the NSAID microspheres is thicker than the enteric coating on the fumaric acid or an ester or a salt thereof (e.g., DMF) microspheres.

It is contemplated that by co-administering the nonsteroidal anti-inflammatory drug (NSAID), such as aspirin and fumaric acid or an ester or a salt thereof (e.g., DMF) to the patient in such a way that the aspirin is absorbed with, or within less than about 5 minutes (or less than about 10 minutes, 15 minutes, 20 minutes, 25 minutes or 30 minutes) after the fumaric acid or an ester or a salt thereof (e.g., DMF), the bioavailability of the fumaric acid or an ester or a salt thereof (e.g., DMF) will be enhanced such that the therapeutically effective dose is about 420 mg/day or less, or about 410 mg/day, or about 400 mg/day, or about 390 mg/day, or about 380 mg/day, or about 370 mg/day, or about 360 mg/day, or between about 360 mg/day and 420 mg/day, or between about 370 mg/day and 410 mg/day, or between 380 mg/day and 400 mg/day.

In some embodiments, the microspheres described herein have an average particle size of less than about 7 mm, or less than about 6 mm, or less than about 5 mm, or less than about 4 mm, or less than about 3 mm, or less than about 2 mm, or less than about 1.7 mm, or less than about 1.6 mm, or less than about 1.5 mm, or less than about 1.4 mm, or less than about 1.3 mm, or less than about 1.2 mm, or less than about 1.1 mm, or less than about 1.0 mm, or less than about 900 μm, or less than about 850 μm, or less than about 800 μm, or less than about 750 μm, or less than about 700 μm, or less than about 650 μm, or less than about 600 μm, or less than about 550 μm, or less than about 500 μm, or less than about 450 μm, or less than about 300 μm. In some embodiments, the particle size ranges from about 900 μm to about 2,000 μm, or from about 850 μm to about 1.7 mm, or from about 1.0 mm to 1.5 mm.

In some embodiments, the microspheres described herein comprise about 80% w/w, or about 75% w/w, or about 70% w/w, or about 65% w/w active ingredient (i.e., aspirin or fumaric acid or an ester or a salt thereof).

### Additional Intraoral Aspirin Portion

In one embodiment, the co-formulation further includes an “intraoral portion” (also referred to as a dissolvable portion) that includes a second amount of nonsteroidal anti-inflammatory drug (NSAID), such as aspirin. This portion of NSAID is formulated such that the NSAID is dissolved quickly in the mouth of a subject and absorbed through the mucosa. By contrast, the portion that includes both NSAID and the fumaric acid or an ester or a salt thereof can be referred to as a “swallowable portion” as it is released in the GI track after being swallowed. The additional NSAID in the intraoral portion, once absorbed through the mucosa, can further reduce the flushing side effect of the fumaric acid or an ester or a salt thereof.

This intraoral portion may be adhered directly to the swallowable portion, or it may be such designed that when it is bitten lightly (e.g. with minimal force, such as the force needed to chew a banana), this intraoral portion breaks off into many pieces within the mouth, and can be chewed and thus absorbed, leaving the harder swallowable portion in the mouth to be swallowed. By making the intraoral portion “crumble” in such a way, the patient will avoid biting hard through the swallowable portion of the tablet, which could be uncomfortable if the swallowable portion is very hard, or could damage the integrity of the swallowable portion, allowing it to be absorbed earlier than desired.

The chewable layer can be formulated, e.g., with a water-soluble sugar and/or a sugar substitutes. Suitable water-soluble sugars and/or sugar substitutes are glucose, maltose, sucrose, dextrose, fructose, sorbitol, mannitol or other types of natural or artificial sweeteners. Mixtures of various sugars or sugar substitutes are also suitable.

The intraoral portion can also be formulated with, e.g., a gel forming agent. Examples of such suitable gel formers are xanthan gum, methylcelluloses such as sodium carboxymethylcellulose or hydroxypropylmethylcellulose, hydroxyethylcellulose, hydroxypropylcellulose, alginates, tragacanth or edible starch. These substances are all commercially available and usually meet the purity requirements and quality regulations for pharmaceutical products. All such gel formers and coatings contemplated are GRAS (generally regarded as safe).

Wetting agents and lubricants such as sodium lauryl sulfate, as well as coloring agents, flavoring agents, sweetening agents (including other nonnutritive sweeteners), tableting agents, stabilizers, antioxidants, cooling agents, and preservatives, can also be present.

A binding agent can also be present such as cellulose, cellulosic derivatives, polyvinyl pyrrolidone, starch, modified starch, and mixtures thereof, and, in particular, microcrystalline cellulose.

In some embodiments, a disintegrant is added to the intraoral portion to allow it to dissolve quickly. Disintegrants are agents added to tablet formulations to promote the breakup of the tablet into smaller fragments in an aqueous environment thereby increasing the available surface area and promoting a more rapid release of the drug substance. Non-limiting examples of disintegrants include pregelatinized starch, microcrystalline cellulose, sodium bicarbonate in combination with citric or tartaric acids, alginic acid, sodium starch glycolate, crospovidone, and Ac-Di-Sol.

In some embodiments, the total dose of NSAID (e.g., aspirin) is present in about a 1:1 ratio between the NSAID microspheres within the swallowable portion and the NSAID in the intraoral portion. In some embodiments, the dose of NSAID (e.g., aspirin) in the intraoral portion is about 20 mg, or about 30 mg, or about 40 mg, or about 50 mg, or about 60 mg, or about 70 mg, or about 75 mg, or about 80 mg. In some embodiments, the dose of NSAID (e.g., aspirin) in the swallowable portion is about 20 mg, or about 25 mg, or about 30 mg, or about 40 mg, or about 50 mg, or about 60 mg, or about 70 mg, or about 75 mg, or about 80 mg.

Other Therapeutic Agents for Co-Formulation with Aspirin

It is contemplated that similar increased bioavailability can be observed for certain other drugs like DMF, such as other therapeutic agents having a niacin-mediated flushing side effect.

In some embodiments, similarly structured co-formulations are disclosed that include nonsteroidal anti-inflammatory drug (NSAID), such as aspirin (in a swallowed portion alone, or in both swallowable and intraoral portions) and a therapeutic agent having a niacin-mediated flushing side effect. The term “therapeutic agent having a niacin-mediated flushing side effect,” as used herein, refers to a group of drugs that activate the nicotinic acid receptor GPR109a, resulting in flushing symptoms commonly observed for patients taking niacin. Sometimes, such agents are also referred to as “nicotinic acid receptor agonists” or “GPR109a agonists.” Non-limiting examples of such therapeutic agents include niacin, nicotyinyl alcohol, acipimox, acifran, newer GPR 109a agonists, hydroxybutyrate, and fumarates (e.g., dimethyl fumarate, mono-ethyl fumarate, diethyl fumarate).

Structure-activity studies have shown common structural features of GPR109a agonists. Some of the GPR109a agonists have a carboxyl group, like in niacin. Another group are anthranilic acid analogs. More of such structural elements are discussed in Boatman et al. J. Med. Chem. 2008; 51(24):7653-62.

### Treatment Methods

In one embodiment, the present disclosure provides a method of treating multiple sclerosis (MS) in a human patient in need thereof. In some embodiments, the disease or condition being treated is one or more of psoriasis, necrobiosis lipoidica, granuloma annulare, sarcoidosis, granulomatous and inflammatory skin disorders, lichen planus pityriasis rubra pilaris, chronic discoid lupus erythematosus, cheilitis granulomatosa, annular elastotic giant cell granuloma, malign melanoma, lupus erythematosus, aplopecia areata, hidradenitis suppurativa, other granulomatous and inflammatory skin disorders, other inflammatory disorders such as colitis, DNA damage in tumor, gastrointestinal ulceration, collagen type II degradation, and other immune modulated diseases.

The method entails, in one embodiment, orally administering to the patient a formulation of any embodiment of the present disclosure. In another embodiment, the method entails orally administering a first amount of nonsteroidal anti-inflammatory drug (NSAID), such as aspirin and a second amount of fumaric acid or an ester or a salt thereof. In some embodiments, the NSAID (e.g., aspirin) is administered at from about 300 mg to about 500 mg per day and the fumaric acid or ester or salt thereof is administered at about 360 mg to about 420 mg per day. In some embodiments, the NSAID (e.g., aspirin) is administered about 1, 2, 3, 5, 10, 15 or 20 minutes after administration of the fumaric acid or an ester or a salt thereof. In some embodiments, the method further entails administering to the patient an additional amount NSAID (e.g., aspirin) to be intraorally absorbed (through the mucosa).

In some embodiments, the patient suffers from relapse-remitting MS (RRMS), a relatively common form of MS. In some embodiments, the patient has a history of non-compliance with a medication due to cutaneous flush or a gastrointestinal side effect. “Non-compliance” as used herein refers to a patient's failure, of at least one time, to take the DMF/MMF medication due to complaint of flushing. In some embodiments, the patient has suspended taking DMF/MMF for at least 1 week, 2 weeks, 1 month, 2 months, 3 months, or 6 months.

In one embodiment, the present disclosure provides a method of treating multiple sclerosis (MS) in a human patient in need thereof.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Intraoral Administration of Aspirin Reduces Fumarate-Induced Flush More than Swallowed Aspirin

Seven human patients with multiple sclerosis who were already taking dimethyl fumarate and had experienced flushing side effects from dimethyl fumarate were recruited for this study. Each patient did not have an allergy or reaction to aspirin or dimethyl fumarate (DMF), had not been diagnosed with kidney disease or liver disease, was not pregnant or planning to be pregnant within the following two months, had not been breastfeeding within the preceding two months, and had not used aspirin for the preceding 7 days.

In Period I, each patient was given their standard dose of 240 mg dimethyl fumarate orally. Each patient was asked to rate his or her flush on the Global Flush Severity Scale (GFSS) (see Paolini et al. Int. J. Clin. Pract. 62(6):896-904 (2008)), when the flush completely resolved. The Global Flushing Severity Score measures, overall, in the previous 24 hours, how each patient rates the flushing symptoms, including redness, warmth, tingling, and itchiness of the skin.

Period II did not start until at least two days upon completion of Period I. At Period II, each patient orally swallowed 162 mg aspirin followed by 240 mg dimethyl fumarate. After the flush completely resolved, then each patient recorded his or her GFSS flush rating.

Not until at least two days later did Period III start. At Period III, each patient was asked to not swallow the orally administered aspirin (162 mg) but to allow the aspirin to be absorbed through the oral mucosa. The aspirin was in powdered form and the remaining aspirin in the mouth was washed out with water. Afterwards, 240 mg of dimethyl fumarate was swallowed with a glass of water. Still, the flush was rated (GFSS) after it was resolved.

The patients during Period III suffered the least severe flush than during any other Periods. Among Periods I through II, the severity of flush was the lowest in Period III (a 52% reduction as compared to Period I), second lowest in Period II (a 33% reduction as compared to Period I) and the highest in Period I. As the total amount of aspirin was the same between Period II and III, this example therefore demonstrates that oral release of aspirin greatly increased aspirin's anti-flushing effect for dimethyl fumarate.

### Example 2. Pilot, Randomized, Open-Label, 2-Way Crossover Comparative Bioavailability Study of Dimethyl Fumarate-Acetylsalicylic Acid 180 mg-150 mg Delayed-Release Capsule (vts-72) and Tecfidera 240 mg Delayed-Release Capsule (Reference) Following a Single Dose in Healthy Subjects Under Fasting Conditions

The objective of this example was to compare the rate and extent of absorption of monomethyl fumarate from a dimethyl fumarate-acetylsalicylic acid 180 mg-150 mg delayed-release capsule (VTS-72) (Test; Treatment A) versus Tecfidera 240 mg delayed-release capsule (Reference; Treatment B), administered as 1×180 mg-150 mg or 1×240 mg delayed-release capsule under fasting conditions.

This was a single center, pilot, comparative bioavailability, open-label, randomized, single-dose, 2-period, 2-sequence, crossover study under fasting conditions. A total of 12 healthy adult male or female volunteers were included in this pilot study. For each period, subjects were confined from at least 10 hours before dosing until 12 hours post-dose. There was a washout of 7 days or more between doses. The washout period could be increased for logistical considerations. Participation of each subject in this study lasted approximately 9 days. Subjects were administered each treatment according to the 2-period, 2-sequence, block randomization scheme.

Treatment A: Subjects were required not to wear dentures or to remove their tongue piercing at the time of dosing. The delayed-release capsules were placed on the subject's tongue. Subjects were instructed to suck the delayed-release capsule until the acetylsalicylic acid coating was dissolved or up to a maximum of 1 minute after the delayed-release capsule had been placed on the subject's tongue. The delayed-release capsule should not be chewed, bitten, or swallowed during that 1-minute period or until the coating is dissolved; only the saliva could be swallowed. The subject was instructed to give a hand sign once the acetylsalicylic acid coating was dissolved (the capsule should feel and taste different). Thereafter, or up to a maximum of 1 minute after the delayed-release capsule was placed on the subject's tongue, 240 mL of water was given to subjects to swallow the capsule. Time of dosing was set to the time the capsule was placed on the tongue. A hand and mouth check was performed to ensure consumption of the medication.

Treatment B: Study medication was administered to each subject and was swallowed whole with 240 mL of water without being sucked, chewed or bitten, and a hand and mouth check was performed to ensure consumption of the medication.

Flushing (including redness, warmth, tingling, and itchiness of the skin) was assessed at 0.5, 1, 1.5, 2, 2.5, 3, 3.5, and 4 hours post-dose using the question and the rating scale presented in Section-Flushing Assessment below. Half grades were not assigned. Flushing symptoms were recorded as adverse events.

GI symptoms were assessed at 10 hours post-dose using the questions and the rating scale presented in Section-Gastrointestinal Symptoms Assessment below. Half grades were not assigned. GI symptoms were recorded as adverse events.

### Flushing Assessment

Flushing was assessed using the question below. The question was asked by the clinical staff:
Question 1: Overall, at this moment, how would you rate your flushing symptoms (including redness, warmth, tingling, and itchiness of the skin)? Score from 0 to 10 (none 0, mild 1 to 3, moderate 4 to 6, severe 7 to 9, extreme 10).

### Gastrointestinal Symptoms Assessment

Gastrointestinal symptoms were assessed using the questions below. Questions were asked by the clinical staff:
Question 2: Overall, during the past 10 hours, how would you rate your GI side effects (nausea, diarrhea, upper abdominal pain, lower abdominal pain, vomiting, indigestion, constipation, bloating, and flatulence)?
Question 3: Overall, during the past 10 hours, how bothersome were your GI side effects (nausea, diarrhea, upper abdominal pain, lower abdominal pain, vomiting, indigestion, constipation, bloating, and flatulence)?

The flushing side effect reported by each of the subject is summarized in Table 1 below. The total reduction of flushing from 15.9 to 7.9 was about 50.3%. When the doses of DMF were normalized to 180 mg in both treatments, the reduction was still 33.6%. Considering that, as shown below, aspirin increased the bioavailability of DMF by about 5%, the reduction of the flushing side effect is actually about 36.8%. Also interestingly, 6 of these 11 subjects had their flushes peak at least 30 mins earlier with VTS-72 (only 2 were later) than with DMF alone.

TABLE 1
Summary of Flush Ratings

Total Flush Rating*

| | Treatment B | Treatment A |
|---|---|---|
| | (240 mg | (180 mg DMF + |
| Subject # | DMF) | 150 mg Aspirin) |
| 1 | (drop out) | (drop out) |
| 2 | 26 | 5 |
| 3 | 8 | 8 |
| 4 | 18 | 14 |
| 5 | 26 | 13 |
| 6 | 14 | 6 |
| 7 | 36 | 13 |
| 8 | 8 | 2 |
| 9 | 11 | 9 |
| 10 | 9 | 0 |
| 11 | 8 | 15 |
| 12 | 11 | 2 |
| Mean | 15.9 | 7.9 |
| Normalized | 11.9 | 7.9 |
| Mean | | |

*Flushes were scored from 0-10 every 30 mins, up to 4 hours post dose (8 total measurements per dose) and added up

Table 2 below provides a descriptive statistics summary of monomethyl fumarate plasma pharmacokinetic parameters.

TABLE 2
Descriptive Statistics Summary of Monomethyl Fumarate Plasma
Pharmacokinetic Parameters

| Parameter | Treatment A | | | | Treatment B | | | |
|---|---|---|---|---|---|---|---|---|
| (units) | N | Mean | SD | CV % | N | Mean | SD | CV % |
| AUC₀₋ₜ | 11 | 3874.01 | 1213.13 | 31.31 | 11 | 4924.24 | 1550.02 | 31.48 |
| (h*ng/mL) | | | | | | | | |
| AUC_{0-inf} | 11 | 3885.63 | 1214.97 | 31.27 | 11 | 4958.99 | 1544.26 | 31.14 |
| (h*ng/mL) | | | | | | | | |
| Residual area | 11 | 0.32 | 0.16 | 49.93 | 11 | 0.74 | 1.79 | 243.23 |
| (%) | | | | | | | | |
| Cₘₐₓ (ng/mL) | 11 | 2034.02 | 599.26 | 29.46 | 11 | 2749.40 | 988.79 | 35.96 |
| T½ₑₗ (h) | 11 | 0.62 | 0.14 | 22.14 | 11 | 0.75 | 0.27 | 35.53 |
| Kₑₗ (/h) | 11 | 1.1618 | 0.2268 | 19.5244 | 11 | 1.0149 | 0.2926 | 28.8326 |
| Correlation | 11 | −0.9978 | 0.0023 | −0.2328 | 11 | −0.9639 | 0.0689 | −7.1439 |
| K_{el Lower} (h) | 11 | 4.768 | 0.959 | 20.105 | 11 | 5.364 | 1.002 | 18.687 |
| K_{el Upper} (h) | 11 | 7.269 | 1.349 | 18.553 | 11 | 8.360 | 1.362 | 16.287 |

When the mean values in both treatments were normalized to 180 mg DMF, the mean values are summarized in Table 3. Co-administration of aspirin did not have significant impact on the Cₘₐₓ of DMF. However, the co-administration of aspirin caused about a 5% increase in AUC₀₋ₜ and AUC_{0-inf}. Also surprisingly, the data showed very tight inter-subject variability (confidence intervals).

TABLE 3
Statistics Summary after Dose Normalization

| Parameter | | Treatment B | Change % |
|---|---|---|---|
| (units) | Treatment A | (normalized) | (A over B) |
| AUC₀₋ₜ | 3874.01 | 3693.18 | 4.90% |
| (h*ng/mL) | | | |
| AUC_{0-inf} | 3885.63 | 3719.2425 | 4.47% |
| (h*ng/mL) | | | |
| Residual | 0.32 | 0.555 | −4.23% |
| area (%) | | | |
| Cₘₐₓ (ng/mL) | 2034.02 | 2062.05 | −1.36% |

Also interestingly, even though the Cₘₐₓ did not have a significant change, the co-administration of aspirin shifted the Tₘₐₓ to about 20 minutes earlier (median). See Table 4.

TABLE 4
Summary Statistics of Tₘₐₓ

| Parameter | Treatment A | | | | Treatment B | | | |
|---|---|---|---|---|---|---|---|---|
| (units) | N | Median | Min | Max | N | Median | Min | Max |
| Tₘₐₓ (h) | 11 | 2.330 | 1.327 | 4.499 | 11 | 2.661 | 0.747 | 4.994 |

This example demonstrates that co-administration of aspirin increased the bioavailability of DMF by about 5% while at the same time reducing the flushing side effect by more than 35%.

### Example 3. Co-Formulation with 205 mg DMF

Example 3 is similar to Example 2 except that the amount of DMF tested was 205 mg. The samples included a dimethyl fumarate-acetylsalicylic acid 205 mg delayed-release capsule (VTS-72) (Test; Treatment A) versus Tecfidera 240 mg delayed-release capsule (Reference; Treatment B), administered under fasting conditions. 16 healthy individuals were enrolled for this study.

The results are presented in Tables 5 and 6 below.

TABLE 5
Descriptive Statistics Summary of Monomethyl Fumarate Plasma
Pharmacokinetic Parameters (analyte: MMF)

| | | | | | | | |
|---|---|---|---|---|---|---|---|
| | | AUC_{0-inf} | Residual | | | | |
| | AUC₀₋ₜ | (hr*ng/ | area | Cₘₐₓ | Tₘₐₓ | T½ | Kₑₗ |
| Statistics | (hr*ng/mL) | mL) | (%) | (ng/mL) | (hr) | (hr) | (1/hr) |

A. Treatment: VTS-72

| | | | | | | | |
|---|---|---|---|---|---|---|---|
| N | 16 | 16 | 16 | 16 | 16 | 16 | 16 |
| Mean | 4122.10 | 4136.42 | 0.54 | 2264.14 | 3.177 | 0.73 | 1.0742 |
| SD | 1298.30 | 1288.64 | 1.02 | 929.02 | 1.101 | 0.35 | 0.3248 |
| Min | 31.50 | 31.15 | 189.55 | 41.03 | 34.646 | 47.25 | 30.2363 |
| Median | 1422.04 | 1481.78 | 0.09 | 631.63 | 1.670 | 0.42 | 0.3672 |
| Max | 4398.64 | 4407.17 | 0.18 | 2406.56 | 3.165 | 0.67 | 1.0303 |
| CV % | 5938.53 | 5950.19 | 4.03 | 3940.65 | 5.000 | 1.89 | 1.6456 |
| Geometric | 3873.08 | 3894.32 | 0.25 | 2050.53 | 2.993 | 0.68 | 1.0185 |
| Mean | | | | | | | |

B. Treatment: Tecfidera

| | | | | | | | |
|---|---|---|---|---|---|---|---|
| N | 16 | 16 | 16 | 16 | 16 | 16 | 16 |
| Mean | 4628.06 | 4636.13 | 0.19 | 2686.31 | 2.802 | 0.61 | 1.2087 |
| SD | 1575.80 | 1577.34 | 0.06 | 1101.89 | 0.872 | 0.14 | 0.2934 |
| Min | 34.05 | 34.02 | 30.27 | 41.02 | 31.120 | 23.81 | 24.2720 |
| Median | 1709.02 | 1714.65 | 0.13 | 757.45 | 1.670 | 0.40 | 0.7842 |
| Max | 4587.13 | 4593.55 | 0.17 | 2577.57 | 2.500 | 0.62 | 1.1221 |
| CV % | 7684.62 | 7695.02 | 0.33 | 4164.55 | 4.500 | 0.88 | 1.7548 |
| Geometric | 4345.39 | 4353.52 | 0.18 | 2437.04 | 2.682 | 0.59 | 1.1762 |
| Mean | | | | | | | |

TABLE 6
Monomethyl Fumarate-Ratios, 90% Geometric Confidence
Intervals, Intra-and Inter-Subject CVs

| | | GEOM | GEOM | | 90% | |
|---|---|---|---|---|---|---|
| | | LS | LS | | Geometric | |
| | Treatment | MEAN | MEAN | | C.I.² | |
| Parameter | Comparison | A | B | Ratio¹ | Lower | Upper |
| AUC₀₋ₜ | Test (A)- | 3881.99 | 4335.42 | 89.54 | 84.88 | 94.46 |
| | Reference | | | | | |
| | (B) | | | | | |
| AUC_{0-inf} | Test (A)- | 3902.50 | 4344.40 | 89.83 | 85.21 | 94.70 |
| | Reference | | | | | |
| | (B) | | | | | |
| Cₘₐₓ | Test (A)- | 2055.19 | 2431.52 | 84.52 | 72.92 | 97.97 |
| | Reference | | | | | |
| | (B) | | | | | |

¹Calculated using least-squares means according to the formula: e^{(DIFFERENCE)} × 100.
²90% Geometric Confidence Interval using In-transformed data.

This example demonstrates that co-administration of aspirin increased the bioavailability of DMF by about 5% while at the same time reducing the flushing side effect by about 61%. Importantly, with such increased bioavailability, the VTS-72 formulation (with 205 mg DMF) would be considered bioequivalent to Tecfidera (240 mg DMF).

### Example 4. DMF with Delayed-Released Aspirin

Example 4 is similar to Examples 2 and 3, except that the enteric coating around the aspirin microspheres are thicker than the DMF, and the DMF dose is about 197 mg. Twenty-four healthy individuals are enrolled. It is believed that the results will show that with this formulation, the bioavailability of DMF will be increased to more than 5%, or that the flush reduction will be further enhanced, or both.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A dosage form comprising:
a first portion comprising a nonsteroidal anti-inflammatory drug (NSAID); and
a second portion comprising a fumaric acid or an ester or a salt thereof,
wherein the first portion and the second portion are formulated such that, upon oral administration to a subject, the first portion and the second portion are released in the gastrointestinal track of the subject, and the NSAID in the first portion is released at substantially the same time as, slower then, or later than the fumaric acid or ester or salt thereof in the second portion.

2. The dosage form of claim 1, wherein the NSAID and fumaric acid or ester or salt thereof are each individually formulated as enterically coated microspheres, and wherein the enteric coating of the NSAID is stronger or thicker than the enteric coating of the fumaric acid or ester or salt thereof.

3. The dosage form of claim 2, wherein the NSAID in the first portion is released at least 1 minute after the release of the fumaric acid or ester or salt thereof in the second portion.

4. The dosage form of claim 1, wherein the NSAID is aspirin.

5. The dosage form of claim 1, wherein the fumaric acid or ester or salt thereof is dimethyl fumarate.

6. The dosage form of claim 5, comprising from about 180 mg to about 210 mg of dimethyl fumarate.

7. The dosage form of claim 1, wherein the first portion comprises from about 20 mg to about 500 mg of aspirin.

8. An enteric coated capsule comprising the dosage form of claim 1.

9. The capsule of claim 8, further comprising a third portion comprising a nonsteroidal anti-inflammatory drug (NSAID) formulated to dissolve in an oral cavity of a subject.

10. The capsule of claim 9, wherein the nonsteroidal anti-inflammatory drug (NSAID) is aspirin.

11. The capsule of claim 10, wherein the third portion comprises from about 20 mg to about 500 mg aspirin.

12. A method of treating multiple sclerosis (MS) in a human patient in need thereof, comprising orally administering to the patient a dosage form of claim 1, twice daily.
